(19) United States
(12) Patent Application Publication
(10) Pub. No.: US 2005/0165450 A1
(43) Pub. Date: Jul. 28, 2005

(54) Passive back extensor device to treat trigger point - back pain

(76) Inventor: Yngrid Perez-Torrens, La Porte, TX (US)

Correspondence Address: JAMES L. JACKSON;James L. Jackson, P.C., 10723 Sugar Hill Dr., Houston, TX, 77042, US

(21) Appl. No.: 11/022,713

(22) Filed: Dec. 27, 2004

Related U.S. Application Data
(60) Provisional application No. 60/533,291, filed on Dec. 30, 2003.

(51) Int. Cl. A61F 5/042; A61H 7/00

(52) U.S. Cl. 606/240; 601/134

(57) ABSTRACT

A passive back extensor device for treatment of trigger-point back pain has a base substrate having upper and lower generally planar surfaces. A plurality of protuberances are integral with the base substrate and project from the upper generally planar surface and are arranged in substantially evenly spaced relation and define a pair of spaced rows so as to collectively define an elongate space between the rows. Each of the protuberances is of generally pyramid configuration having a rounded end oriented for passive manual pressure engagement with the paraspinal muscles along an individual's back on each side of the spine and with the elongate space receiving the bony protuberances of the spine. Each of the protuberances is arranged with one of its corners located adjacent a side edge or an end edge of the base substrate so that the planar surfaces and base is oriented in angular relation with the side and end edges of the base substrate.

### RELATED PROVISIONAL APPLICATION

Applicant hereby claims the benefit of U.S. Provisional Patent Application No. 60/533,291, filed on Dec. 30, 2003 by Yngrid Perez-Torrens and entitled “Passive Back Extensor Device To Treat Trigger Point—Back Pain”.

### BACKGROUND OF THE INVENTION

1. Field of the Invention

The present invention generally relates to the therapeutic treatment of back pain based on Trigger Point by Travell and Simmon and Back Extension by McKenzie Therapy. Particularly this invention enables an individual to alleviate trigger point associated tissue pain and thereby permit the individual to improve body posture, promoting back flexibility.

2. Description of the Prior Art

Back pain and particularly low back pain is the most common of all complaints whose cause is more difficult to find due to the many times it is part of a referred symptom. Low back pain is the most common cause of occupational disability in industrial societies. Pain problems may be related to either the neuromusculoskeletal systems or noneuromuscoskeletal systems. The source of pain could be cutaneuos, deep somatic pain, visceral pain, or referred pain. As Classified by Kanner R M: Low Back Pain. Pain Management: Theory and Practice, Philadelphia, 1996, F A Davis, pp 126-144, Low back pain is classified in Acute pain originating in the bony element or muscle, aponeurosis, and nerves. Chronic low back pain could be somatic nociceptive, neuropathic and psycogenic pain. There are many studies, theories, and tools created to treat back pain. Most Treatments appears to be directed at pain relief.

According to Robin McKenzie, in studies described on the “Lower Lumbar Spine: Mechanical Diagnosis and Therapy” Spinal Publications 1981 (reprinted 1998), McKenzie states that the patient must learn to manage future back pain by using a self-treatment and self-manipulative method. The studies also indicate that treatment of back pain, for optimum beneficial results, must be implemented during an attack of low back pain rather than after the back pain has subsided, in order to educate or teach prophylactic methods to the patient. These types of patients typically do not suffer from serious pathology and they often respond satisfactorily to treatment. It is often that will the recurrence of low back pain becomes progressively more severe with each successive attack, thus the patient will benefit significantly from timely treatment.

Sitting posture, loss of extension range and frequency of flexion are the three predisposing factors in the etiology of low back pain exposed by McKenzie. Relaxed sitting for any length of time places the lumbar spine in a fully stretched position. This will become painful, if maintained for a prolonged period. It had been demonstrated that when a patient is sitting, the intradiscal pressure increases as the spine moves into kyphosis, and decreases as it moves into lordosis. Postural factors other than sitting may predispose the patient to low back pain. Some sleeping positions and work-related postures may be potentially damaging or perpetuate low back pain also. A reduced range of extension influences the posture in sitting, standing and walking. A frequency of flexion characteristics of life-style on western cultures in the twentieth century can cause incremental stress on the posterior annular wall and cause the fluid nucleus to move posteriorly.

Low back pain can occur by precipitating factors such as movement and lifting. It is often the unexpected and unguarded movement that causes a sudden episode of low back pain. This movement may occur during work related activities, be it domestic or occupational, and in sports and recreational activities.

On the other hand, lifting produces a strain that is often a precipitating factor, especially when heavy, prolonged and repeated lifting are involved. It has been studied and described that lifting from the forward bent position is one of the most stressful activities. When a certain weight is lifted with the back bent and the knees straight, the intradiscal pressure rises up to five times compared with that present when standing erect. However, when the same weight is lifted with the back straight and the knees bent there is a marked reduction in intradiscal pressure. The intradiscal pressure decreases as it moves into lordosis or back extension.

In addition to the sitting position and other predisposing factors in the etiology of low back pain are the loss of back extension and the frequency of flexion. Repeated and sustained flexions stress the nucleous and force it to move posteriorly, and if the disk loses its hydrostatic mechanism, could cause a disk protrusion or herniation of the nucleus pulposus with nerve root compression. Back extension, as explained in details by McKensie, reduce the stress on the posterior annular wall and simultaneously cause the fluid nucleus to move anteriorly. Any current anatomy book typically explains in detail about the intervertebral disk and components.

It is known that back extension helps to reduce back pain. There are many tools used to provide support and back extension, for example: Hazard Rowland, U.S. Pat. No. 4,981,131 describes a method using a fluid-inflatable bag positioned in the back at the lumbar area to provide cyclic mobilization of the lumbar spine of a person. These changes of inflation and deflection of gas or other fluid moves the spine of the person through a range of degrees of lordosis. This invention provides cyclic passive mobilization of the lumbar spine of a person while seated or supine. Waldron, et al., U.S. Pat. No. 5,702,333, introduced a method and a concave surface device to passively extend a human's lumbar spine when the human lays with her or his stomach on top surface. Another invention that combines pain relief with back extension is set forth by Summers, Neil in U.S. Pat. No. 6,041,457. Summers et al present a backrest device having a supporting surface presenting two elongate continuous or discontinuous protuberances which extend alongside one another and are arranged to engage the back of a person, resting in use against the supporting surface, on each side of the spine, with a channel between the protuberances to accommodate the bony part of the spine with substantially no pressure on the bony part of the spine.

The contraction of a small part of the fiber of a muscle is defined as a “Highly irritable localized spot of exquisite tenderness in a nodule in a palpable taut band of muscle tissue.”, i.e., “trigger point”. Some of the activities and events that create trigger points are accidents, falls, strains, and overwork. For instance when one carries excessive loads, exercises vigorously when out of condition, or hammers away too long and too hard at some unaccustomed work, trigger point pain is often experienced.

Constant muscle abuse has taken on a number of different names, such as overuse syndrome, repetitive motion injury, repetitive strain, cumulative trauma disorder, and occupational myalgia. When muscles are overcontracted or overstretched they can be expected to result in trigger points. Trigger points are always painful when manual pressure is applied to them. A trigger point feels like a knot or a small lump that can range in size from a pinhead to a pea. In the long muscles a trigger point feels like a short piece of cord easily mistaken for a tendon.

Travell and Simons concluded that a trigger point exists when overstimulated sarcomeres become unable to release their contracted state. The part of a muscle fiber that actually does the contracting is a microscopic unit called a sarcomere. Contraction occurs in a sarcomere when its two parts come together and interlock like fingers. Millions of sarcomeres have to contract in the muscles to cause the body to make even the smallest movement. A trigger point exists when over stimulated sarcomeres are chemically prevented from releasing from their interlocked state. When sarcomeres in a trigger point hold their contraction, blood flow essentially stops in the immediate area. The resulting oxygen starvation and accumulation of the waste products of metabolism irritate the trigger point. The trigger point responds to this emergency by sending out pain signals until the brain institutes a policy of rest for the muscle.

There are in the market several devices uses to treat muscle trigger points and also there are several of them patented as invention. Not all of them attribute to trigger point physiology, but all of them function as pain reliever devices. For instance: Taylor, Walter U.S. Pat. No. 6,589,143 introduced a patent of a portable back pain-relieving device for use by a person while the person is sitting or reclining comprising. This back pain reliever has a linear alignment support that runs along the spine and with at least two bi-pad mounted on opposite sides of the spine of a person. The application of pressure directly over a trigger point will cause an increment of pain, which in most cases will be initially quite severe.

Contraindications:

The following contraindications should be considered: These contraindications will be the same as in McKenzie Therapy, due to the fact that the device of the present invention promotes back extension. Consequently, a device constructed according to the principles of the present invention cannot be used for an individual where back extension is contraindicated, or for whom no position or movement can be found to reduce or centralize pain patterns. If the individual's back pain initially is not reduced with any position or movement, he or she is unsuited for mechanical therapy, at least at that time. Also, the use of this device is contraindicated if serious pathology is suspected and where there has been no apparent reason for the onset of symptoms; or if this pain has been present for many weeks or months, and during that time increasing in intensity. Those individuals who exhibit signs of extreme pain whose spine freezes or immobilizes when palpation is attempted are considered unsuitable, at least during this stage. Finally, this device is contraindicated for use directly over a scar tissue not well healed, and over damaged or irritated skin. It is advisable, for comfort, to use the passive back extension device of the present invention through a layer of clothing.

### SUMMARY OF THE INVENTION

It is a principal feature of the present invention to provide a novel passive back extensor device for self-use by individuals to treat trigger point back pain;

It is another feature of the present invention to provide a novel passive back extensor device that is of integral construction and thus has no parts that can become separated and lost when the device is stored between self-use treatments;

It is a feature of the present invention to provide a novel passive back extensor device that can be easily and efficiently self-used by a person experiencing trigger point back pain, at the time that the pain is being experienced, thus obviating the need to schedule a treatment appointment with a therapist;

It is also a feature of the present invention to provide a novel passive back extensor device that is composed of soft, yieldable rubber or polymer material and thus has virtually no potential for causing injury during use, handling or storage; and

Another feature of the present invention is the provision of a novel passive back extensor device that is easily handled and transported by the user.

Briefly, the invention presented in this specification is also based on the Physiology of trigger point back pain which is explained by Janet G. Travell, M.D and David G. Simons, M.D. in their 1992 manual entitled “Myofascial Pain and Dysfunction: The Trigger Point Manual. Vol. II, 2nd ed. Baltimore: Lippincott, Williams and Wilkins.

According to the present invention, a back extensor device for trigger point treatment is built in a particular shape to produce a pointy constant pressure to a painful trigger point during a passive back extension to treat back pain and bad posture, as well as to provide lumbar support while sitting. A device embodying the principles of the present invention includes a substrate which is preferably flexible, but may be rigid or somewhat rigid if desired. From the substrate projects a plurality of protuberances which may be integral with the substrate or may be fixed to the substrate and are arranged in spaced rows. Each protuberance defines a curved or somewhat pointed distal end that is oriented for contact with the body of a person and also defines a proximal end that is joined with or projects from the substrate.

In use the back extensor device is, pressed between the body of an individual engaged in self-treatment and a flat surface, enable the individual himself or herself to discover and treat trigger points located on the back and in the most acute part of his/her vertebral spinal curve. For comfort, that part of the users body to be treated with the back extensor device should be covered by a layer of clothing. Trigger points are typically sharply painful when compressed, and to gain benefits the effect of pressure on the trigger points must “hurt good”, that is to create a significant level of pain at or about a particular trigger point. If an individual has a really bad trigger point, he/she should try initially applying significant mechanical pressure, thus giving it an initial shot of pain, then backing off, and waiting ten seconds or so before proceeding on with the treatment. The idea is to work deeper with far less discomfort. It also works producing back extension.

A normal response to reduce back pain is its centralization from its most distal extent. This implies that the pain is moving towards or centered in the lumbar area. It is advisable to see a specialist in this case to rule out fractures, tumors, infection or any other spinal pathology or referred condition where back extension is contraindicated. If it is known that the pain is a symptom for bad posture or prolonged body position, the back extensor device of the present invention will work in one or few sessions. The device must be placed first between T10-L2 spinal vertebras. The pointy parts or finger-like protuberances of this device are separated to enter in contact with the paraspinal muscle and where the trigger points are located. This device can be used when an individual is sitting or in supine lying position. The pointy protuberances of this device will simulate fist-like positioning of a person's hand wherein the middle finger is extended somewhat and forms a protruding knuckle. During treatment by a therapist this protruding knuckle is caused to press on the paraspinal muscle with sufficient manual pressure or force to cause relaxation of trigger points that are identified. During self-use of the passive back extensor device of the present invention, the pointy protuberances are positioned with the small rounded tips of the protuberances in contact with the muscle while the individual being self-treated is sitting or is lying down in supine position. The device is supported during such self-treatment by any suitable surface, such as the surface of a bed, a floor surface or floor mat, the back of a chair, etc. Manual pressure or force is achieved by controlled movement of the user's body to achieve as little or as much manual pressure as is needed to cause a tolerable level of tissue pain at detected trigger points to result in efficient and satisfactory treatment.

An advantage of the invention is its portability and that the patient can utilize this invention whenever a tissue pain arises. A feature of the invention is that the patient can apply the mechanical treatment as long as it seems beneficial. No longer is the individual treatment dependent on the time slot available to him or her in the physical therapist's office to alleviate the pain. For instance, It is very beneficial to those individuals that spend long hours sitting doing office work. The passive back extensor device of the present invention is preferably provided in the form of a durable one piece or integral device having a substrate, with a number of protuberances projecting from the substrate. Each of the protuberances preferably has a fairly large base at its intersection with the substrate and tapers from the base to a rounded point.

This invention is a device built in a particular shape to produce a pointy constant pressure to a painful trigger point during a passive back extension to treat back pain and bad posture, as well as to provide lumbar support while sitting. This device pressed between the body and a flat surface enable an individual himself or herself to discover and treat trigger points located on his back and in the most acute part of his/her vertebral spinal curve. For comfort this device should be used through a layer of clothing. Trigger points hurt when compressed, and to gain benefits the effect of pressure must “hurt good”. If an individual has a really bad trigger point, he/she should try giving it an initial shot of pain, then backing off, and waiting ten seconds before going on. The idea is to work deeper with far less discomfort. It also works producing back extension. A normal response to reduce back pain is its centralization from its must distal extend. This implies that the pain is moving towards or centered in the lumbar area. It is advisable to see a specialist to rule out in this case fractures, tumors, infection or any other spinal pathology or referred condition where back extension is contraindicated. If it is known that the pain is a symptom for a bad posture or prolonged position, this device will work in one or few sessions. The device must be placed first between T10-L2 spinal vertebras. The pointy parts of this device are separated to enter in contact with the paraspinal muscle and where the trigger points are located. This device can be used when an individual is sitting or in supine lying position or even when the person is standing and leaning with the device engaging the person's back and supported by a wall or other such support. The pointy protuberances of this device will simulate application of a finger knuckle to the user's back muscle, causing pressure over the paraspinal muscle while the individual in sitting or lying down in supine position. An advantage of the invention is its portability and that the patient can utilize this invention whenever a tissue pain arises. A feature of the invention is that the patient can apply passive mechanical treatment as long as it seems beneficial to the user and most importantly at a time when trigger point back pain is being experienced. No longer is the individual treatment dependent on the time slot available to him or her in the physical therapist's office to alleviate the pain. The passive back extensor device is a durable one piece device so that no part of it can become separated and lost. The device provides direct distraction forces to preselected areas of the spine, and in particular, to the lumbar spine producing decompression or unloading in the intervertebral discs and facet joints. The passive back extensor device is easy to use, facilitates placing of the patient for treatment. It is very beneficial to those individuals that spend long hours sitting doing office work, driving vehicles and heavy equipment and those that lack time for regular exercise.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the preferred embodiment thereof which is illustrated in the appended drawings, which drawings are incorporated as a part hereof.

It is to be noted however, that the appended drawings illustrate only a typical embodiment of this invention and are therefore not to be considered limiting of its scope, for the invention may admit to other equally effective embodiments.

In the Drawings:

FIG. 1 is an isometric illustration, showing a passive back extensor device embodying the principles of the present invention and being seen from above, with six pyramid shaped pointed protuberances projecting upwardly from a generally rectangular base or substrate;

FIG. 2 a side elevational view showing the passive back extensor device passive back extensor device of FIG. 1;

FIG. 3 is a pictorial illustration representing the hand of a person, such as a therapist, with the fingers bent to form a fist and showing the central finger protruding to form an extended knuckle of generally pyramid shape in position for manually applying pressure to a trigger point of a patient;

FIG. 4 is a plan view showing the passive back extensor device of FIGS. 1 and 2, and showing the arrangement, configuration and relative positioning of the pyramid shaped pointed protuberances on the rectangular base substrate;

FIG. 5 is an isometric illustration showing positioning of the passive back extensor device of FIGS. 1, 3 and 4, with pyramid shaped protuberances thereof projecting downwardly;

FIG. 6 is a side elevational view of the passive back extensor device of the present invention showing a bent form of the device to indicate the flexibility thereof;

FIG. 7 is a side elevational view of the passive back extensor device of the present invention showing opposite bending of the device, as compared with FIG. 6 to indicate the flexibility thereof; and

FIG. 8 is an end view of the passive back extensor device of the present invention showing another bent form of the device, as compared with FIGS. 6 and 7 to further identify the flexibility thereof and illustrating its capability of readily conforming to a yieldable or non-yieldable support substrate.

### DETAILED DESCRIPTION OF THE INVENTION

Referring now to the drawings and first to FIG. 1, a passive back extensor device constructed according to the principles of the present invention is shown generally at 10. The passive back extensor device is provided for application of controlled mechanical pressure to trigger points that may exist in the paraspinal muscles that are situated along both sides of the spinal column of a patient's back and particularly in the region of the lower lumbar spine, where back pain is typically described as “low back pain”. The passive back extensor device 10 is preferably of integral construction, being composed entirely of soft rubber or any of a number of relatively soft and flexible rubber-like polymer materials. It should be borne in mind however, that the passive back extensor device 10 may be constituted as an assembly of a number of different materials if desired.

The passive back extensor device has a generally rectangular and generally flat base substrate 12 which is preferably flexible and has a thickness from about 0.3 cm to about 0.5 cm and preferably has a thickness of about 0.4 cm. The base substrate 12 defines an upper or top generally planar surface 13 and a lower or bottom generally planar surface 15. Typically, during use of the passive back extensor device, the lower generally planar surface 15 will be in supported contact with the upper surface of a bed or pad or a surface of a chair back, thus permitting support of the device as therapy is being conducted. The generally rectangular base substrate 12, which may be of other configuration without departing from the spirit and scope of the present invention, has a length of about 18 cm and a width of about 12.2 cm. The length and width of the generally rectangular base substrate 12 may vary to slightly longer or shorter and slightly narrower or wider if desired to accommodate the musculoskeletal differences of adult patients. The flexibility of the generally rectangular base substrate 12 is indicated in the photographs of FIGS. 6, 7 and 8. Thus, if the base substrate is supported by a rather soft support, such as the mattress of a bed or the back of a padded chair, pressure being transferred from the body of a patient to the trigger point passive back extensor device 10 will likely cause bending of the generally rectangular base substrate either longitudinally, transversely or both, thus essentially conforming the device 10 to the body configuration of the patient's back.

A plurality of protuberances 14 project from a generally planar surface of the base substrate 12 and define rounded ends 24 that contact the user during self-conducted therapy. These protuberances can be of rectangular or circular cross-sectional configuration if desired, assuming that they do not become bent as they are positioned in force transmitting contact with or over the selected muscles of the user. The protuberances 14 are preferably generally pyramid shaped and are preferably integral with the generally rectangular base substrate 12. The protuberances project upwardly from the upper, generally planar surface 13 of the base substrate 12 to a height of about 6 cm. At the juncture of the protuberances with the upper surface 13 of the base substrate 12, each of the generally pyramidal shaped protuberances 14 have a base measurement of about 4 cm on each of the four sides 16, 18, 20 and 22 and taper upwardly to form a generally pyramidal four sided configuration. The generally pyramid configuration of the protuberances 14 enhance the structural integrity of the protuberances in the region of their juncture with the base substrate 12 and yet permit the end portions of the protuberances to be flexible and resilient due to the soft and flexible material from which the protuberances are composed.

The upper portions of each of the generally rectangular tapered protuberances 14 are defined by rounded upper end surfaces 24, giving each of the generally rectangular protuberances 14 a somewhat pointed configuration. These small rounded upper end surfaces enhance manual application of force, i.e., pressure to the small “trigger points” of the paraspinal muscles of a user, typically in the region of the vertebral spinal curve. Preferably, the terminal extent of each of the rounded ends intersect or have coincidence with an imaginary plane “P” being substantially parallel with the upper surface 13 of the base substrate 12. Also, since the base substrate is preferably composed of a flexible material, it can easily be bent longitudinally and/or laterally, thus permitting the user to alter the relative orientation of the protuberances as is evident in FIGS. 6-8.

The respective sides 16, 18, 20 and 22 of each of the protuberances 14 are preferably defined by inclined, upwardly converging substantially planar surfaces that each merge with a rounded upper end surface 24. It should be borne in mind however, that the specific configuration of the protuberances 14 is not intended to be limited by the representation shown in the drawings. Protuberances of other configuration, such as conical or cylindrical, may be employed as well, assuming that they each have a somewhat pointed configuration at the ends which is defined by rounded or somewhat pointed end surfaces.

The preferred embodiment of the present invention is a passive back extensor device having six, substantially evenly spaced pyramidal projections or protuberances each having small substantially rounded or curved upper surfaces 24 for engagement with the painful trigger points of the back muscles. The protuberances are arranged in two spaced rows and are typically oriented to engage the muscles at three spaced locations on each side of the spinal vertebras. Preferably, the generally pyramid shaped protuberances 14 are arranged with opposed corners thereof oriented so as to coincide with imaginary lines that are substantially parallel with the side edges 23 and 25 of the base substrate 12 and substantially parallel with the longitudinal center-line C/L of the base substrate 12. This arrangement minimizes the potential for bending of the protuberances either longitudinally or laterally of the device 10 as manual pressure is employed to urge the protuberances into forceful engagement with designated muscle regions of the user. The longitudinal center-line C/L intersects end edges 27 and 29 of the generally rectangular base substrate 12 at the respective center points thereof.

During treatment, the device 10 is positioned so as to provide direct distraction forces to preselected areas of the spine, particularly the areas where back pain is especially prominent due to the presence of trigger points in the muscle tissue. With the rounded end surfaces of the pyramidal protuberances in engagement with appropriate areas of the muscles, manual pressure is applied to urge the small rounded end surfaces of the protuberances against the skin over the muscle tissue where the trigger points are found. A patient can accomplish this activity by locating the device with the rounded ends of the protuberances engaging the patient's back and with the rectangular base engaging the back of a chair or the upper surface of a conventional bed or floor pad. If significant pain is not initially experienced, then an indication is provided that one or more trigger points have not been contacted. In this case, the device is shifted upwardly, downwardly or sidewise and manual pressure is again applied. This activity is typically repeated until the user experiences significant localized pain, thus indicating that a trigger point has been encountered. At this point, manual pressure is applied to the extent that the user can tolerate, sometimes relaxing and then periodically reapplying the pressure to cause a particular trigger point to normalize or release the trigger point and thereby eliminate or minimize the back pain that was previously experienced by the user.

The pyramid shaped protuberances are preferably composed of soft resilient material such as soft rubber or a soft, rubber-like polymer. Preferably, the pyramid shaped protuberances are integral with the base substrate 12, though they may separately formed or manufactured and then attached to the base substrate. For example, it may be desirable to provide a passive back extensor device having soft tapered protuberances that are mounted on a base substrate of less flexible material. Thus, when dissimilar materials are used for manufacturing the passive back extensor device, the protuberances can be bonded or otherwise mounted to the base substrate. Though the base substrate 12 is preferably of flexible nature as evidence by FIGS. 6-8, it may take the form of a rather rigid member of rectangular or other desired configuration, with the soft, flexible protuberances 14 mounted to it in any suitable fashion. Though the protuberances are shown to have four inclined sides, it should be borne in mind that the protuberances may have more or less sides and, if desired, may be of substantially conical configuration or substantially cylindrical configuration with rounded upper end surfaces.

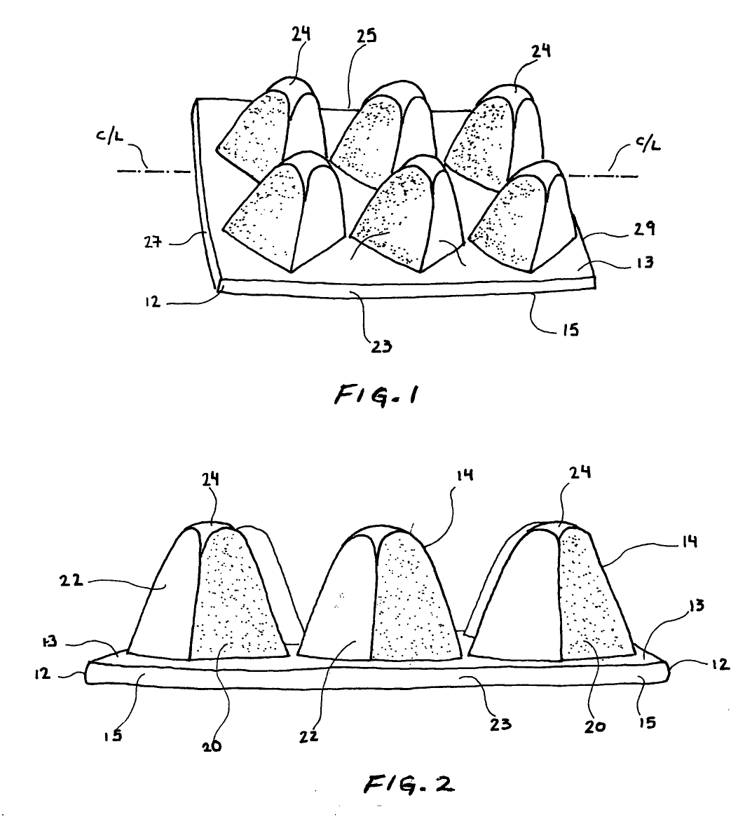

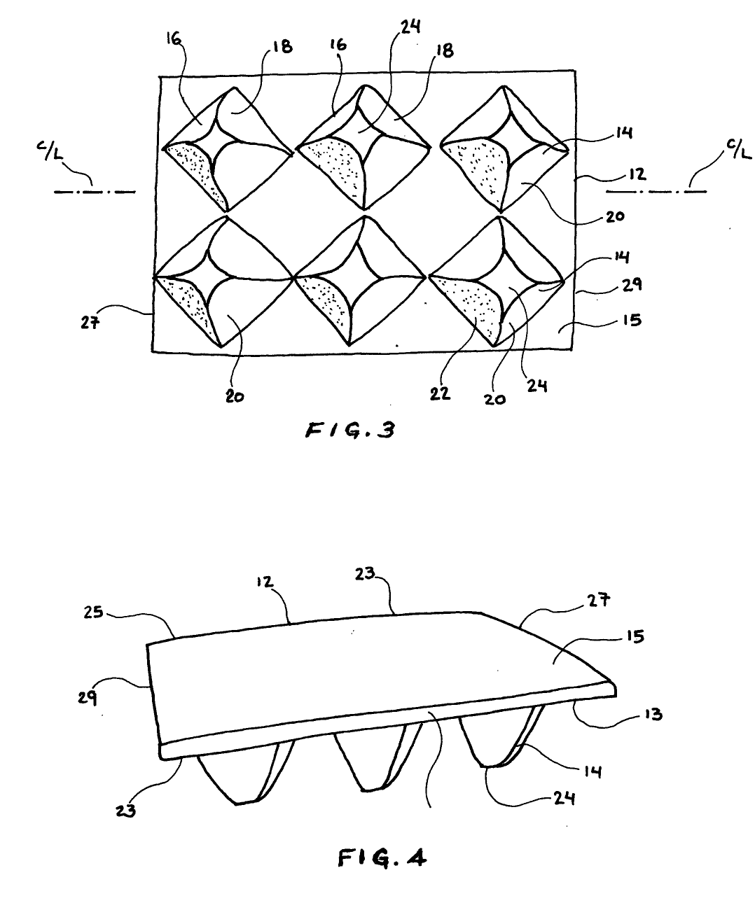

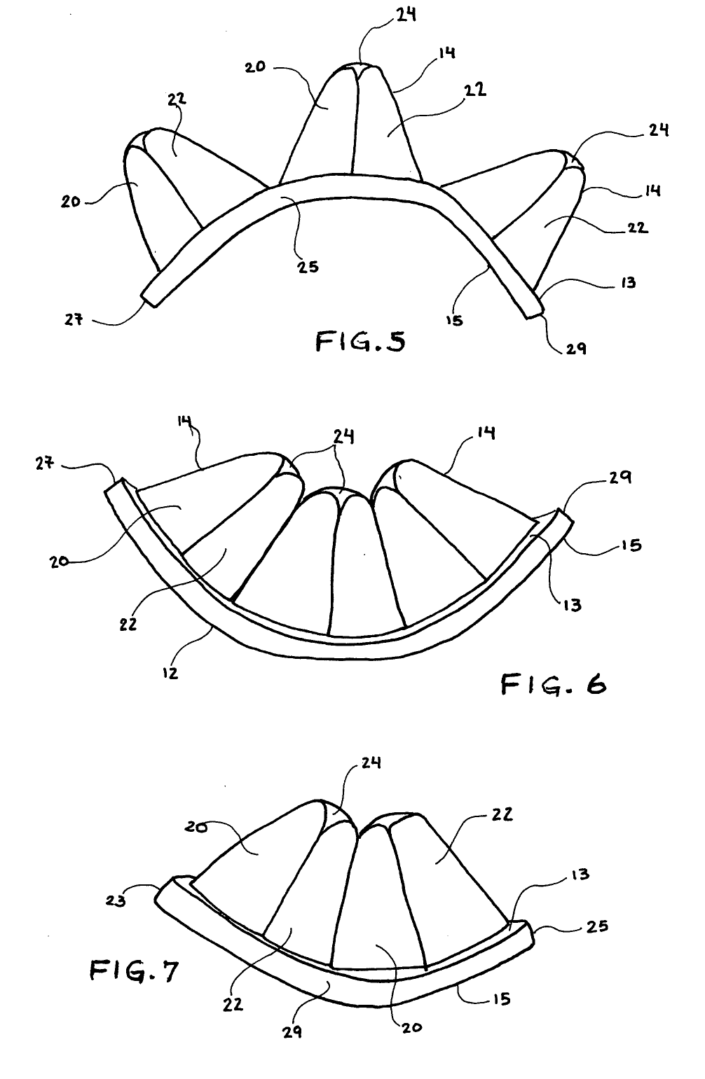

The passive back extensor device 10 of the present invention is preferably made in one piece of (soft rubber) with 6 protuberances of generally pyramidal shape projecting from the base substrate and having rounded ends 24 to simulate knuckles such as are formed by the fist-like bent fingers of a person's hand as shown in FIG. 2a. Typically, when trigger point treatment is being conducted by a therapist, the therapist will bend his/her fingers to form a fist, with the central finger projecting and forming a small knuckle region. The therapists hand is then manipulated to cause the projecting knuckle to apply manual pressure to a trigger point that has been found in the muscle tissue.

The pyramid shaped protuberances are distributed on the base substrate 12 in two spaced rows, with three protuberances to each row and are substantially evenly spaced on a generally flat base substrate and evenly spaced from the longitudinal center-line C/L of the base substrate. Each pointy protuberance end 24 will have substantially the same separation or spacing to permit efficient engagement with the paraspinal muscle on each side of the spine and with a space between the rows, i.e., along the longitudinal center-line of the device, provided for receiving the bony paraspinal protuberances of the user, without applying mechanical pressure to the paraspinal protuberances.

The invention is typically used with a layer of clothing interposed between the ends of the pyramid shaped protuberances and the skin of the patent to ensure the comfort of the patient. The individual using the passive back extensor device 10 must expect a significant increment of pain in the area of pressure when treatment is being initiated. The trigger points virtually always hurt when compressed, and to gain benefits of the treatment the effect of pressure must cause significant pain at the trigger points, i.e., “hurt good”. The threshold of pain should approach the maximum that the individual can tolerate, this being different for each individual since the tolerable thresholds of pain are different for each individual. If an individual has a really bad trigger point, he/she should try giving it an initial shot of pain, by applying protuberance pressure to the trigger point, then backing off, and waiting ten seconds or so before going on. The idea is to work deeper into the muscles with far less discomfort. As the trigger point begins to relax or normalize as the result of passive pressure treatment, the level of pain will diminish significantly, thus providing indication of successful treatment. The device also works producing back extension. The extension must be maintained for no more than about 15 minutes.

As mentioned above, the pyramid shaped protuberances are preferably composed of soft resilient material such as soft rubber or a soft, rubber-like polymer. The device can be manufactured by pouring uncured, substantially liquid rubber or polymer material into a mold and permitting the material to cure within the mold. The device is complete when it is removed from the mold as an integral unit having a base substrate structure 12, with a plurality of substantially pointed protuberances being integral with the base substrate structure and projecting upwardly from it. An alternative embodiment of the present invention may be manufactured by providing a base substrate structure that may be flexible or rigid as desired. To this base substrate is then affixed a plurality of rather soft and resilient, pointed protuberances that may be of pyramid shaped form or of conical configuration, with rounded upper surfaces for engagement with the trigger points in the muscle mass on each side of the spine of a patient. The passive back extensor device may have a number of other forms without departing from the spirit and scope of the present invention.

### USE OF THE INVENTION

The user will locate the device on any suitable generally flat surface (floor, mat, or bed) and at the level of the expected T10-L2 regions of the spine. The individual, from the sitting position, will lie down slowly having his/her back bending and extending vertebra by vertebra, starting from his/her sacrum and maintaining abdominal muscle contraction at all times so as to achieve the supine position slowly and in precisely controlled manner. Once his/her back is in contact with the device and the head is down completely and in contact with the bed or other support, the individual should try to relax and remain in this position for no more than 15 minutes or as long as can be tolerated. At this point the individual will be experiencing considerable pain as the pointed or rounded ends 24 of the protuberances 14 apply manual pressure to the trigger points of the muscles. It is advisable to try breathing in/out deeply and slowly. The individual could adopt a knees bent position to increase more pressure on the trigger points, as well as crossing upper extremities over his/her chest, so as to maximize the weight that will be pressing down on the protuberances of the passive back extensor device 10.

To move from the supine or lying position the individual could bend his/her knees and roll to one side, or simply reverse the movement from sitting to supine with the knees extended, keeping the abdominal muscles in a state of controlled contraction at all times. Once the individual has removed the device and she/he will have returned to the sitting position and then must complete the self-treatment session with stretching exercises of slow movement. The individual should stretch the paraspinal muscle by trying to reach his/her toes with the individual's fingers and with the knees extended. Then, the individual could sit down and stretch his/her trunk rotator muscles to the right and to the left.

Another very complex back stretching exercise could be called spinal twist. To do the spinal twist to right the individual will be in sitting position with left lower extremities extended and with right leg crossed over the left knee and with the foot of the right leg flat on the floor and with the knees bent. Then with the left upper extremity crossing over the right knee, the individual will hold his/her left knee moving slowly. His/her right upper extremity will cross back to reach the left hip if possible. The individual doing this spinal twist position will try to look back and far up to the left, moving slowly while inhaling to stretch and holding this position a few seconds as tolerable to keep stretching a little more. Each stretching exercise can be repeated twice if the individual desires it. Then last stretching called rocking movement to start the cycle with the device will be with the knees bent in a supine position. The individual will hold abdominal contraction at all times, with both knees bent with his/her upper extremities crossing in as hugging both lower extremities and from this position, rock several times to get to a sitting position. The passive back extensor device 10 can be used several times in succession and each use, for maximum beneficial results, must be followed by one or all of the stretching exercises here explained.

When the passive back extensor device 10 is used while the individual is in a sitting position in a chair it will require only one or two stretching exercises. The device is easily located in the general area of the back to be treated (in the, up, or down T10-L2 area) by bending the trunk forward and inserting the device between the users back and the chair back, with the base substrate in supported contact with the chair back. When the user leans back, the rounded ends of the protuberances 14 will engage the back at the paraspinal muscles. The passive back extensor device 10 is then shifted up, down or laterally until significant pain is encountered, thus indicating that one of the rounded ends 24 of the protuberances 14 is located at a trigger point. The user will then lean back further, thus manually forcing the protuberance or protuberances into the muscle mass, thus applying manual pressure to the trigger point. The user can easily control the amount of force or pressure that is applied to the trigger point, simply by controlled movement of the torso. The user can easily apply manual pressure to the trigger point to the extent of toleration of the level of pain that occurs, and then by forward movement of the torso, decrease the manual pressure and relieve or lessen the pain. The user can then wait 10 to 15 seconds or so and then repeat the procedure. As trigger point normalization occurs the user will notice that the level of pain will have decreased markedly, indicating successful self-treatment.

Immediately after treatment, it is highly desirable that the user accomplish stretching of the paraspinal muscles to minimize the potential for re-establishment of the previously treated trigger points. The stretching exercises after using the device in sitting position could be as follows: After removing the device from between the user's back and the chair back, the user can bend the torso forward, folding down vertebra by vertebra from head to sacrum and simultaneously relax the head, arms and shoulders. The user can then sit back slowly, maintaining the abdominal muscles in a contracted state. A second stretching exercise could start at either side, with the user moving the torso far back following with the arms and head trying to touch or see the back support. The individual will maintain the lower extremities in a straight line with knees bent at an angle of about 90°. This cycle could be repeated to the extent that the individual can tolerate.

In view of the foregoing it is evident that the present invention is one well adapted to attain all of the objects and features hereinabove set forth, together with other objects and features which are inherent in the apparatus disclosed herein.

As will be readily apparent to those skilled in the art, the present invention may easily be produced in other specific forms without departing from its spirit or essential characteristics. The present embodiment is, therefore, to be considered as merely illustrative and not restrictive, the scope of the invention being indicated by the claims rather than the foregoing description, and all changes which come within the meaning and range of equivalence of the claims are therefore intended to be embraced therein.

1. A passive back extensor device for treatment of trigger-point back pain, comprising:
a base substrate having upper and lower generally planar surfaces and defining a longitudinal center-line;
a plurality of protuberances projecting from said upper generally planar surface and being disposed in substantially evenly spaced relation and being substantially evenly spaced from said longitudinal centerline and collectively defining an elongate space; and
each of said plurality of protuberances having a rounded end oriented for passive manual pressure engagement with an individual's back on each side of the individual's spine and with said elongate space receiving the bony protuberances of the individual's spine.

2. The passive back extensor device of claim 1, comprising:
said base substrate being composed of flexible material and permitting longitudinal and lateral bending thereof and controlling relative orientation of said plurality of protuberances.

3. The passive back extensor device of claim 1, comprising:
said plurality of protuberances being integral with said base substrate.

4. The passive back extensor device of claim 1, comprising:
each of said plurality of protuberances being of generally pyramid configuration having a generally rectangular base joined with said base substrate and having substantially planar inclined sides each joined with adjacent sides and each merging with said rounded end.

5. The passive back extensor device of claim 1, comprising:
each of said plurality of protuberances being of tapered upwardly converging configuration having a base joined with said base substrate and having inclined sides merging with said rounded end.

6. The passive back extensor device of claim 1, comprising:
said plurality of protuberances being integral with said base substrate and each being of tapered upwardly converging configuration having a base joined with said base substrate and having inclined sides merging with said rounded end and having said rounded ends thereof each intersecting an imaginary plane being substantially parallel with said upper surface of said base substrate.

7. The passive back extensor device of claim 1, comprising:
each of said plurality of protuberances being of generally pyramid configuration having a generally rectangular base joined with said base substrate and having substantially planar inclined sides each joined with adjacent sides and each merging with said rounded end; and
said rounded ends of said plurality of protuberances each intersecting an imaginary plane being substantially parallel with said upper surface of said base substrate.

8. The passive back extensor device of claim 7, comprising:
said base substrate being flexible longitudinally and laterally and when bent causing change of the relative positions of said plurality of protuberances.

9. A passive back extensor device for treatment of trigger-point back pain, comprising:
a base substrate having upper and lower generally planar surfaces and defining a longitudinal center-line;
a plurality of protuberances projecting from said upper generally planar surface and being disposed in substantially evenly spaced relation and being substantially evenly spaced from said longitudinal centerline and collectively defining an elongate space along said longitudinal center-line, each of said plurality of protuberances being of generally pyramid configuration having a generally rectangular base joined with said base substrate and having substantially planar inclined sides each joined with adjacent sides; and
each of said plurality of protuberances having a rounded end oriented for passive manual pressure engagement with an individual's back on each side of the individual's spine and with said elongate space receiving the bony protuberances of the individual's spine, each of said substantially planar inclined sides merging with said rounded end.

10. The passive back extensor device of claim 9, comprising:
said base substrate being of generally rectangular configuration having substantially parallel side edges; and
said substantially planar inclined sides of each of said plurality of protuberances defining corners, one of said corners of each of said plurality of protuberances being located adjacent to and in substantially equally spaced relation with said substantially parallel side edges and said substantially planar inclined sides of each of said plurality of protuberances being oriented in angular relation with said substantially parallel side edges.

11. The passive back extensor device of claim 10, comprising:
said base substrate having substantially parallel end edges;
said plurality of protuberances being arranged in spaced rows, each row having endmost protuberances; and
one of said corners of said plurality of said endmost protuberances being located adjacent to and in substantially equally spaced relation with said substantially parallel end edges and said substantially planar inclined sides of each of said plurality of protuberances having angular orientation with respect to said end edges.

12. The passive back extensor device of claim 9, comprising:
each of said plurality of protuberances being of generally pyramid configuration having a generally rectangular base joined with said base substrate and having substantially planar inclined sides each joined with adjacent sides and each merging with said rounded end.

13. The passive back extensor device of claim 9, comprising:
each of said plurality of protuberances being of tapered upwardly converging configuration having a base joined with said base substrate and having inclined sides merging with said rounded end.

14. The passive back extensor device of claim 9, comprising:
said plurality of protuberances being integral with said base substrate and each being of tapered upwardly converging configuration having a base joined with said base substrate and having inclined sides merging with said rounded end and having said rounded ends thereof each intersecting an imaginary plane being substantially parallel with said upper surface of said base substrate.

15. The passive back extensor device of claim 9, comprising:
each of said plurality of protuberances being of generally pyramid configuration having a generally rectangular base joined with said base substrate and having substantially planar inclined sides each joined with adjacent sides and each merging with said rounded end; and
said rounded ends of said plurality of protuberances each intersecting an imaginary plane being substantially parallel with said upper surface of said base substrate.

16. The passive back extensor device of claim 15, comprising:
said base substrate being flexible longitudinally and laterally and when bent causing change of the relative positions of said plurality of protuberances.

17. A passive back extensor device for treatment of trigger-point back pain, comprising:
a generally rectangular flexible base substrate having upper and lower generally planar surfaces and defining a longitudinal center-line and generally parallel side edges and generally parallel end edges;
a plurality of protuberances being integral with said base substrate and projecting from said upper generally planar surface, said plurality of protuberances being arranged in spaced rows and being substantially evenly spaced from said longitudinal centerline and collectively defining an elongate space along said longitudinal center-line, each of said plurality of protuberances being of generally pyramid configuration having a generally rectangular base joined with said base substrate and having substantially planar inclined sides each joined with adjacent sides and defining corners; and
each of said plurality of protuberances having a rounded end oriented for passive manual pressure engagement with an individual's back on each side of the individual's spine and with said elongate space receiving the bony protuberances of the individual's spine, each of said substantially planar inclined sides merging with said rounded end.

18. The passive back extensor device of claim 17, comprising:
said substantially planar inclined sides of each of said plurality of protuberances defining corners, one of said corners of each of said plurality of protuberances being located adjacent to and in substantially equally spaced relation with said substantially parallel side edges and said substantially planar inclined sides of each of said plurality of protuberances being oriented in angular relation with said substantially parallel side edges.
said base substrate having substantially parallel end edges;
said plurality of protuberances being arranged in spaced rows, each row having endmost protuberances; and
one of said corners of said plurality of said endmost protuberances being located adjacent to and in substantially equally spaced relation with said substantially parallel end edges and said substantially planar inclined sides of each of said plurality of protuberances having angular orientation with respect to said end edges.
